(19) United States
(12) Patent Application Publication
(10) Pub. No.: US 2010/0240923 A1
(43) Pub. Date: Sep. 23, 2010

(54) ENZYMATIC SYNTHESIS OF (METH)ACRYLIC ESTERS OF HYDROXY-FUNCTIONAL AROMAS

(75) Inventors: Dietmar Haering, Neu-Edingen (DE); Gabi Winter, Wachenheim (DE); Arnold Schneller, Seeheim-Jugenheim (DE); Francesca Aulenta, Mannheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/743,819

(22) PCT Filed: Nov. 29, 2007
(86) PCT No.: PCT/EP07/63014

§ 371 Date: May 20, 2010

(51) Int. Cl. C07C 69/52; C12P 7/62

(52) U.S. Cl. 560/225; 435/135

(57) ABSTRACT

A process for preparing (meth)acrylic esters (F) of hydroxy-functional aromas (A), in which at least one hydroxy-functional aroma (A) in the presence of at least one enzyme (E) is esterified with (meth)acrylic acid (S), or transesterified with at least one (meth)acrylic ester (D), the reaction in the case of the transesterification being effected in the absence of solvents.

The present invention relates to a process for preparing (meth)acrylic esters of hydroxy-functional aromas and to their use.

In the context of the present invention, (meth)acrylic acid is understood to mean acrylic acid and/or methacrylic acid; (meth)acrylic esters are understood to mean acrylic esters and/or methacrylic esters.

(Meth)acrylic esters are prepared usually by acid- or base-catalyzed esterification of (meth)acrylic acid or transesterification of other (meth)acrylic esters with alcohols.

(Meth)acrylic esters of hydroxy-functional aromas are known in principle. Such esters are also known as so-called fragrance acrylates and find use, for example, as a comonomer for slow-release fragrance polymers. Such slow-release fragrance polymers are understood to mean those polymers which release the fragrance slowly and in a controlled manner.

Athawale et al. disclose, in Journal of Molecular Catalysis B: Enzymatic 16 (2001, 169-173), the enzymatic synthesis of chiral menthyl methacrylates. The preparation was achieved by enantioselective transesterification of (±)-menthol with different lipases in solvents, and the reactants used were methyl methacrylate, vinyl methacrylate or 2,3-butanedione monooxime acrylate. The influence of various parameters was investigated, for example the influence of the temperature, type and amount of the catalyst and different solvents. The best conversion rates were achieved with diisopropyl ether as the solvent.

In Tetrahedron Letters 43 (2002), 4797-4800, Athawale et al. describe the enzyme-catalyzed preparation of geranyl methacrylate by transesterification. This is effected by reacting geraniol with 2,3-butanedione monooxime acrylate in a solvent with different lipases as catalysts. In this document, Athawale et al. disclose in particular that the selection of a suitable solvent is essential for biocatalytic reactions. Here too, diisopropyl ether is described as the most suitable solvent with which the highest conversion rates are achieved.

In Biotechnology Progress 19 (2003), 298-302, Athawale et al. likewise describe the influence of reaction parameters on lipase-catalyzed transesterification for the preparation of citronellyl methacrylate. The transesterification is effected starting from methyl methacrylate, vinyl methacrylate or 2,3-butanedione monooxime acrylate in the presence of solvents. In this publication, Athawale et al. follow the preceding publications, according to which the transesterification is effected in an organic solvent, for example diisopropyl ether.

The syntheses disclosed in the prior art take place in the presence of solvents, diisopropyl ether being used as the preferred solvent. To date, the influence of different solvents on the reaction rates has been examined. Such solvents have to be removed again from the mixture in a complicated manner after the reaction has ended, in order that the resulting (meth)acrylic esters of hydroxy-functional aromas can be polymerized, for example to prepare fragrance acrylates. Moreover, residual traces of solvent can change the aroma or the odor in an undesired manner.

It was therefore an object of the present invention to provide a process with which (meth)acrylic esters of hydroxy-functional aromas can be obtained by (trans)esterification. The process should give rise to purities of at least >99% without complicated purification steps such as extraction or distillation of the product.

The object is achieved by a process for preparing (meth)acrylic esters (F) of hydroxy-functional aromas (A), in which at least one hydroxy-functional aroma (A) in the presence of at least one enzyme (E) is esterified with (meth)acrylic acid (S), or transesterified with at least one (meth)acrylic ester (D), the reaction in the case of the transesterification being effected in the absence of solvents.

Hereinafter, the reactants (meth)acrylic acid (S) and (meth)acrylic ester (D) are also summarized together under the term (meth)acrylic compound (B).

With the aid of the process according to the invention, the preparation of such (meth)acrylic esters (F) is possible in high chemical and space-time yield and under mild conditions while dispensing with protecting group operations and using simple starting materials. Especially in the case of the transesterification, the complicated removal of a solvent is dispensed with, so that the resulting (meth)acrylic esters (F) can be polymerized directly to prepare fragrance acrylates.

Hydroxy-functional aromas (A) suitable in accordance with the invention are those alcohols which comprise at least one hydroxyl group and which can be perceived with odor receptors, either directly through the nose (nasal perception) or via the pharyngeal cavity when eating or drinking (retronasal perception).

The hydroxy-functional aromas (A) may comprise from one to six, preferably from one to four, more preferably from one to three, even more preferably from one to two hydroxyl groups, and in particular exactly one hydroxyl group.

The hydroxy-functional aromas (A) usable in accordance with the invention may also comprise other heteroatoms, for example nitrogen, oxygen and sulfur; they are preferably formed only from carbon, hydrogen and oxygen atoms.

The hydroxy-functional aromas (A) usable in accordance with the invention may also comprise other functional groups, for example C—C double bonds, amino, carboxyl, ether or carboxylic ester groups.

The hydroxyl groups of the hydroxy-functional aromas (A) usable in accordance with the invention may be primary, secondary or tertiary; preference is given to those having primary or secondary hydroxyl groups and particular preference to those having primary hydroxyl groups.

Primary hydroxyl groups are hydroxyl groups which are bonded to a carbon atom which is bonded to exactly one further carbon atom. Analogously, in secondary hydroxyl groups, the carbon atom bonded to it is correspondingly bonded to two carbon atoms, and, in the case of tertiary hydroxyl groups, to three carbon atoms.

Preferred hydroxy-functional aromas (A) are primary alcohols of the general formula (I):

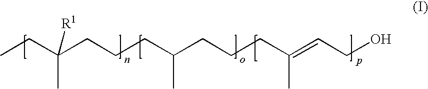

in which n, o and p are each integers of from 0 to 10 in each case, with the proviso that at least one of the variables n, o or p is at least 1, and in which the particular monomer units which are bracketed by the variables n, o and p are present in any sequence, and R¹ is selected from hydrogen, hydroxyl and C₁-C₁₀-alkyl, C₂-C₁₀-alkenyl and C₂-C₁₀-alkynyl.

The variables n, o and p are preferably integers of from 0 to 8, more preferably from 0 to 6, even more preferably from 0 to 4 and in particular from 0 to 2, in each case with the proviso that at least one of the variables n, o or p is at least 1.

The total number of monomer units which arises from the sum of n, o and p is preferably not more than 10, more preferably not more than 6, even more preferably not more than 4 and in particular not more than 2.

The particular monomer units which are bracketed by the variables n, o or p may be present in any sequence, so that, for example, the monomer unit which bears the functional hydroxyl group may be either a C═C double bond (monomer unit with the variable p) or a C—C single bond (monomer unit with the variable o), each of which may optionally bear a substituent R¹ (monomer unit with the variable n).

R¹ in the monomer unit with the variable n is selected from hydrogen, hydroxyl, C₁-C₁₀-alkyl, C₂-C₁₀-alkenyl and C₂-C₁₀-alkynyl.

In the context of the present invention, C₁-C₁₀-alkyl is understood to mean straight-chain or branched hydrocarbon radicals having up to 10 carbon atoms, for example methyl, ethyl, propyl, isopropyl, n-butyl, sec-butyl, tert-butyl, 1,1-dimethylethyl, pentyl, 2-methylbutyl, 1,1-dimethylpropyl, 1,2-dimethylpropyl, 2,2-dimethylpropyl, 1-ethylpropyl, hexyl, 2-methylpentyl, 3-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, 2,4,4-trimethylpentyl, 1,1,3,3-tetramethylbutyl, nonyl and decyl, and isomers thereof. Preference is given to alkyl radicals having from 1 to 6 carbon atoms.

C₂-C₂₀-Alkenyl is understood to mean unsaturated, straight-chain or branched hydrocarbon radicals having from 2 to 10 carbon atoms and a double bond in any position, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, and also the isomers of heptenyl, octenyl, nonenyl and decenyl. Preference is given to alkenyl radicals having from 2 to 6 carbon atoms.

In the context of the present invention, C₂-C₁₀-alkynyl are straight-chain or branched hydrocarbon groups having from 2 to 10 carbon atoms and a triple bond in any position, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl, and also the isomers of heptynyl, octynyl, nonynyl, decynyl. Preference is given to alkynyl radicals having from 1 to 6 carbon atoms.

R¹ in the monomer unit with the variable n is preferably hydrogen, hydroxyl or C₁-C₁₀-alkyl, more preferably hydrogen, hydroxyl or C₁-C₆-alkyl, and more preferably hydrogen or hydroxyl.

It will be appreciated that the R¹ radical in a plurality of monomer units with the variable n may have the same or different definitions.

The particular monomer unit which is bracketed by the variable n, o or p is based on an isoprene unit. Such acyclic isoprenoids (also known as terpenoids) belong to a large group of natural substances which usually have a pleasant aromatic odor, and whose content of carbon atoms is usually a multiple of 5 (isoprene rule). The carbon skeleton can be formed by simple head-to-tail bonding from isoprene units.

Particularly preferred hydroxy-functional aromas (A) of the general formula (I) are summarized in Table 1.

TABLE 1
Particularly preferred hydroxy-functional aromas of the general formula (I)

| | | Chemical |
|---|---|---|
| Structural formula | Name | name |
| 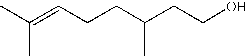 | citronellol | 3,7- dimethyl- oct-6-en-1- ol |
| 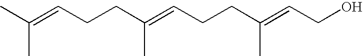 | farnesol | 3,7,11,- trimethyl- dodeca- 2,6,10- trien-1-ol |
| 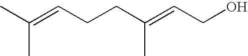 | geraniol | 3,7- dimethyl- octa-2,6- dien-1-ol |
| 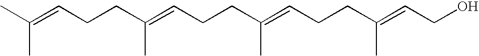 | geranylgeraniol | 3,7,11,15- tetramethyl- hexadeca- 2,6,10,14- tetraen-1-ol |
| 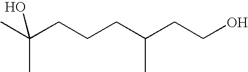 | hydroxycitronellol (hydroxyciol) | 3,7- dimethyl- octane-1,7- diol |
| 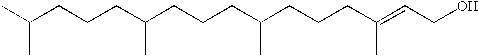 | phytol | 3,7,11,15- tetramethyl- hexadec-2- en-1-ol |
| 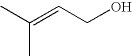 | prenol | 3-methyl- but-2-en- 1ol |
| 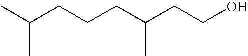 | tetrahydrogeraniol | 3,7- dimethyl- octane-1-ol |

Very particular preference is given to citronellol, geraniol, hydroxyciol, phytol, prenol and tetrahydrogeraniol.

However, it is also possible in principle to use hydroxy-functional aromas (A) with a primary hydroxyl group, which do not comprise a base structure composed of monomer units based on isoprene. Examples of such representatives are compiled in table 2.

TABLE 2
Hydroxy-functional aromas (A) which do not comprise a base structure
composed of monomer units based on isoprene

| Structural formula | Name | Chemical name |
|---|---|---|
| 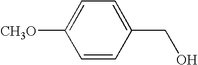 | anise alcohol | 4-methoxy- benzyl alcohol |
| 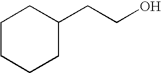 | cyclohexylethanol | 2-cyclohexyl- ethanol |
| 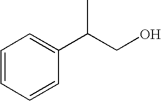 | hydratopic alcohol | 2-phenyl- propan-1-ol |
| 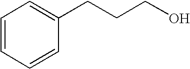 | hydroxycinnamyl alcohol | 3-phenyl- propan-1-ol |
| 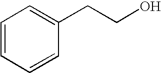 | phenylethyl alcohol | 2-phenyl- ethanol |
| 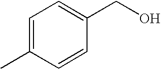 | p-tolyl alcohol | 4-methyl- benzyl alcohol |
| 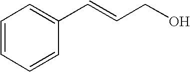 | cinnamyl alcohol | 3-phenylprop- 2-en-ol |

Particularly preferred hydroxy-functional aromas (A) which do not comprise a base structure composed of monomer units based on isoprene are anise alcohol, cyclohexyl alcohol, hydroxycinnamyl alcohol and cinnamyl alcohol.

In addition to hydroxy-functional aromas with primary hydroxyl groups, it is also possible in principle to use hydroxy-functional aromas which have a secondary or tertiary hydroxyl group. However, the (trans)esterification in these cases is often more difficult, since the aromas are sterically more demanding. Preference is therefore also given to hydroxy-functional aromas (A) with a secondary hydroxyl group.

Suitable hydroxy-functional aromas (A) with secondary or tertiary hydroxyl groups are summarized in Table 3.

| | | Chemical |
|---|---|---|
| Structural formula | Name | name |
| 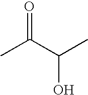 | acetoin (acetylmethylcarbinol) | 3-hydroxy- butan-2-one |
| 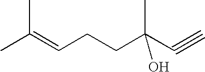 | dehydrolinalool | 3,7- dimethyl-oct- 1-yn-6-en-3- ol |
| 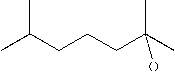 | dimethylheptanol | 2,6- dimethyl- heptan-2-ol |
| 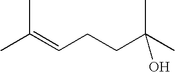 | dimethylheptenol | 2,6- dimethyl- hept-5-en-2- ol |
| 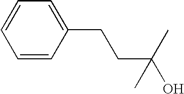 | dimethylphenyl- ethylcarbinol | 2-methy1-4- phenyl- butan-2-ol |
| 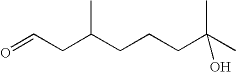 | hydroxycitronellal | 3,7- dimethyl-7- hydroxy- octan-1-al |
| 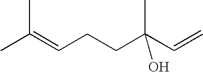 | linalool | 3,7- dimethyl- octa-1,6- dien-3-ol |
| 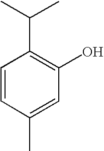 | menthol | 2-isopropyl- 5-methyl- phenol |
| 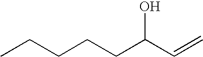 | morrilol | oct-1-en-3-ol |
| 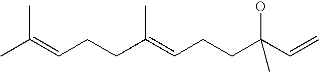 | nerolidol | 3,7,11- trimethyl- dodeca- 1,6,10-trien- 3-ol |
| 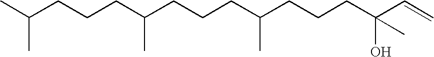 | iso-phytol | 3,7,11,15- tetramethyl- hexadec-1- en-3-ol |
| 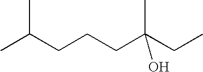 | tetrahydrolinalool | 3,7- dimethyl- octan-3-ol |

Preferred hydroxy-functional aromas (A) with a secondary hydroxyl group are acetoin, menthol and morrilol. Very particular preference is given to morrilol.

When the hydroxy-functional aromas (A) mentioned are optically active, they are preferably used in racemic form or as diastereomer mixtures, but it is also possible to use them as pure enantiomers or diastereomers or as enantiomer mixtures.

In the reaction step, the esterification with (meth)acrylic acid (S) or preferably the transesterification of the alcohol (A) is effected with at least one (meth)acrylic ester (D) in the presence of at least one enzyme (E), preferably one which catalyzes the transesterification.

(Meth)acrylic acid (S) can be used for the esterification, or (meth)acrylic esters (D) of a saturated alcohol for the transesterification, preferably saturated C₁-C₁₀-alkyl esters or C₃-C₁₂-cycloalkyl esters of (meth)acrylic acid, more preferably saturated C₁-C₄-alkyl esters of (meth)acrylic acid.

In the context of this document, saturated means compounds without C—C multiple bonds (except of course the C═C double bond in the (meth)acryloyl units).

Examples of (meth)acrylic esters (D) are the methyl, ethyl, n-butyl, isobutyl, n-octyl and 2-ethylhexyl esters of (meth)acrylic acid, 1,2-ethylene glycol di- and mono(meth)acrylate, 1,4-butanediol di- and mono(meth)acrylate, 1,6-hexanediol di- and mono(meth)acrylate, trimethylolpropane tri(meth)acrylate and pentaerythritol tetra(meth)acrylate.

Particular preference is given to the methyl, ethyl, n-butyl and 2-ethylhexyl esters of (meth)acrylic acid.

Enzymes (E) usable in accordance with the invention are, for example, selected from hydrolases (E.C. 3.-.-.-) and among these particularly from the esterases (E.C. 3.1.-.-), lipases (E.C. 3.1.1.3), glycosylases (E.C. 3.2.-.-) and proteases (E.C. 3.4.-.-), in free form or in chemically or physically immobilized form on a support, preferably lipases, esterases or proteases and more preferably esterases (E.C. 3.1.-.-). Very particular preference is given to Novozyme® 435 (lipase from Candida antarctica B) or lipase from Alcaligenes sp., Aspergillus sp., Mucor sp., Penicilium sp., Geotricum sp., Rhizopus sp., Burkholderia sp., Candida sp., Pseudomonas sp., Thermomyces sp. or porcine pancreas; especially preferred lipases are those from Candida antarctica B or from Burkholderia sp.

The enzyme content in the reaction medium is generally in the range from about 0.1 to 10% by weight, based on the alcohol (A) used.

The enzymatic (trans)esterification of (meth)acrylic acid(s) or of methacrylic esters (D) is effected generally at from 0 to 100° C., preferably from 20 to 80° C., more preferably from 20 to 70° C., even more preferably from 20 to 60° C. and especially preferably from 20 to 40° C.

The reaction time depends upon factors including the temperature, the amount used and the activity of the enzyme catalyst, and on the required conversion, and also on the hydroxy-functional aroma (A). The reaction time is preferably adjusted such that the conversion of the hydroxyl functions present in the hydroxy-functional aroma (A) to be converted, i.e. the hydroxyl functions with a relatively low level of substitution, is at least 70%, preferably at least 80%, more preferably at least 90%, even more preferably at least 95%, in particular at least 97% and especially at least 98%. In general, from 1 to 72 hours, preferably from 3 to 36 hours and more preferably from 3 to 24 hours are sufficient for this purpose.

The molar ratio of (meth)acrylic acid compound (B) (based on the (meth)acryloyl units) to hydroxy-functional aroma (A) (based on hydroxyl groups) can be set within a wide range, for example in a ratio of from 100:1 to 1:1, preferably from 50:1 to 1:1, more preferably from 20:1 to 1:1 and most preferably from 10:1 to 1:1.

According to the invention, the transesterification of (meth)acrylic esters (D) with at least one hydroxy-functional aroma (A) is performed in the absence of solvents. This is especially advantageous because the complicated removal of the solvent after the reaction has ended is dispensed with, and the resulting (meth)acrylic ester (F) can thus be processed further directly, for example for the preparation of slow-release fragrance acrylates.

The esterification of (meth)acrylic acid (S) can be performed in the presence of a solvent, but preference is given to not adding a solvent for the reasons mentioned. The mixtures are generally substantially anhydrous (i.e. water addition below 10% by volume, preferably below 5% by volume, more preferably below 1% by volume and most preferably below 0.5% by volume).

Suitable organic solvents for the esterification are those known for these purposes, for example tertiary monools such as C₃-C₆-alcohols, preferably tert-butanol, tert-amyl alcohol, pyridine, poly-C₁-C₄-alkylene glycol di-C₁-C₄-alkyl ether, preferably polyethylene glycol di-C₁-C₄-alkyl ether, for example 1,2-dimethoxyethane, diethylene glycol dimethyl ether, polyethylene glycol dimethyl ether 500, methyl tert-butyl ether, ethyl tert-butyl ether, C₁-C₄-alkylene carbonates, especially propylene carbonate, C₃-C₆-alkyl acetates, especially tert-butyl acetate, tetrahydrofuran, toluene, 1,3-dioxolane, acetone, isobutyl methyl ketone, ethyl methyl ketone, 1,4-dioxane, tert-butyl methyl ether, cyclohexane, methylcyclohexane, toluene, hexane, dimethoxymethane, 1,1-dimethoxyethane, acetonitrile, and mono- or polyphasic mixtures thereof. It may be advantageous to remove water released by means of a binary heteroazeotrope which boils very close to the temperature optimum of the enzyme (E) used.

Optionally, aqueous solvents can be added to the organic solvents, so as to form—depending on the organic solvents—mono- or polyphasic reaction solutions. Examples of aqueous solvents are water and aqueous, dilute (from 10 to 100 mM) buffers, for example with a pH in the range from about 6 to 8, for example potassium phosphate or TRIS-HCl buffer.

The water content in the reaction mixture is generally 0-10% by volume. Preference is given to using the reactants without pretreatment (drying, water doping).

The substrates are present in the reaction medium in dissolved form, suspended as solids or in emulsion. The initial concentration of the reactants is preferably in the range from about 0.1 to 20 mol/l, in particular from 0.15 to 10 mol/l or from 0.2 to 5 mol/l.

The reaction can be effected continuously, for example in a stirred reactor or in a stirred reactor battery, or batchwise.

The reaction can be performed in all reactors suitable for such a reaction. Such reactors are known to those skilled in the art. Preference is given to effecting the reaction in a stirred tank reactor or a fixed bed reactor.

To mix the reaction mixture, any processes may be used. Specific stirrer apparatus is not required. The reaction medium may be mono- or polyphasic and the reactants are dissolved, suspended or emulsified therein, if appropriate initially charged together with the molecular sieve, and admixed with the enzyme preparation at the start of the reaction, and, if appropriate, once or more than once in the course of the reaction. The temperature is adjusted to the desired value during the reaction and can, if desired, be increased or decreased during the course of the reaction.

When the reaction is performed in a fixed bed reactor, the fixed bed reactor is preferably equipped with immobilized enzymes, in which case the reaction mixture is pumped through a column filled with the enzyme. It is also possible to perform the reaction in a fluidized bed, in which case the enzyme is used immobilized on a support. The reaction mixture can be pumped continuously through the column, in which case the residence time and hence the desired conversion are controllable with the flow rate. It is also possible to pump the reaction mixture through a column in circulation, in which case it is also possible to simultaneously distil off the alcohol released under reduced pressure.

The removal of water in the case of an esterification or alcohols which are released from the alkyl (meth)acrylates in a transesterification is effected continuously or stepwise in a manner known per se, for example by distillation, vacuum, azeotropic removal, absorption, pervaporation and diffusion through membranes.

Suitable methods for this purposes are preferably molecular sieves or zeolites (pore size, for example, in the range of about 3-10 angstrom), or a removal by distillation or with the aid of suitable semipermeable membranes.

However, it is also possible to feed the removed mixture of alkyl (meth)acrylate and the parent alcohol thereof, which frequently forms an azeotrope, directly into a plant for preparing the alkyl (meth)acrylate, in order to reutilize it there in an esterification with (meth)acrylic acid.

After the reaction has ended, the reaction mixture obtained from the (trans)esterification can be used further without further purification or it can be purified in a further step if required.

In general, in one reaction step, only the enzyme (E) used is removed from the reaction mixture, and the reaction product, in the case of the esterification, is removed from any organic solvent used.

A removal from the enzyme is effected generally by filtration, absorption, centrifugation or decantation. The enzyme removed can subsequently be used for further reactions.

In the case of the esterification, the removal of the organic solvent is generally effected by distillation, rectification or, in the case of solid reaction products, by filtration.

If appropriate, the reaction mixture can be purified if desired, for example by filtration, distillation, rectification, chromatography, treatment with ion exchangers, adsorbents, neutral, acidic and/or alkaline scrubbing, stripping or crystallization.

However, in the purification step, preference is given to removing only the enzyme used and any solvent used, or the excess (meth)acrylic acid or (meth)acrylate.

According to the invention, apart from a removal of the enzyme catalyst, however, no additional purification step is required, especially when no solvents are added.

The reaction conditions in the enzymatic (trans)esterification are mild. Owing to the low temperatures and other mild conditions, the formation of by-products during the reaction is prevented, which might otherwise stem, for example, from chemical catalysts or result from undesired free-radical polymerization of the (meth)acrylate used, which can otherwise only be prevented through addition of stabilizers.

In the inventive reaction, additional stabilizers may be added to the (meth)acrylic compound (B) over and above the storage stabilizer present in any case, for example hydroquinone monomethyl ether, phenothiazine, phenols, for example 2-tert-butyl-4-methylphenol, 6-tert-butyl-2,4-dimethylphenol or N-oxyls such as 4-hydroxy-2,2,6,6-tetramethylpiperidine N-oxyl, 4-oxo-2,2,6,6-tetramethylpiperidine N-oxyl, for example in amounts of from 50 to 2000 ppm. Advantageously, the (trans)esterification is performed in the presence of an oxygenous gas, preferably air or air-nitrogen mixtures.

The present invention further provides the (meth)acrylic esters (F) obtained from the hydroxy-functional aromas (A) by enzymatic (trans)esterification. These are notable especially in that they generally comprise less than 1.0% by-products from rearrangement reactions of the multiple bond from acid- or base-catalyzed side reactions. The advantage of the (meth)acrylic esters (F) thus obtained by the process according to the invention is that, owing to the aroma present therein, they are suitable for preparing so-called slow-release fragrance acrylates. These slow-release fragrance acrylates release the fragrance, i.e. the aroma, in a slow and controlled manner.

Such slow-release fragrance acrylates can be used in all sectors in which a pleasant fragrance is desired. Fields of use are, for example, washing compositions, cleaning compositions, adhesives, for example carpet adhesives, and disperse dyes.

For the preparation of such slow-release fragrance acrylates, the inventive (meth)acrylic esters (F), as a monomer or as a comonomer, are subjected to a polymerization with other ethylenically unsaturated compounds, so as to obtain homopolymers of (meth)acrylic esters (F) or copolymers with other ethylenically unsaturated compounds. The collective term co(polymers) is therefore also used hereinafter when both homo- and copolymers are meant.

Copolymers of (meth)acrylic esters (F) as a comonomer and other ethylenically unsaturated compounds as a main monomer consist of the so-called main monomers preferably to an extent of at least 40% by weight, more preferably to an extent of at least 60% by weight, most preferably to an extent of at least 80% by weight.

The main monomers are selected from monoethylenically unsaturated C₃-C₆-carboxylic acids, C₁-C₂₀-(meth)acrylic esters, -(meth)acrylamides and -(meth)acrylonitriles, vinyl esters of carboxylic acids comprising up to 20 carbon atoms, vinyl esters of carboxylic acids having from 1 to 20 carbon atoms, vinylaromatics having up to 20 carbon atoms, vinyl halides, vinyl ethers of alcohols comprising from 1 to 10 carbon atoms, aliphatic, optionally halogenated hydrocarbons having from 2 to 8 carbon atoms and 1 or 2 double bonds, open-chain N-vinylamide compounds, vinylidenes or mixtures of these monomers.

Preferred monoethylenically unsaturated C₃-C₆-carboxylic acids are, for example, acrylic acid, methacrylic acid, crotonic acid, fumaric acid, itaconic acid, maleic acid and their C₁-C₂₀-alkyl esters, amides, nitriles and anhydrides, for example methyl acrylate, ethyl acrylate, methyl methacrylate, ethyl methacrylate, n-butyl acrylate, n-butyl methacrylate, aryl methacrylates, acrylic anhydride, itaconic anhydride, monomethyl maleate, dimethyl maleate, monoethyl maleate, diethyl maleate, 2-ethylhexyl acrylate, 2-ethylhexyl methacrylate, maleic anhydride and its monoesters, alkylene glycol (meth)acrylates, acrylamide, methacrylamide, N-dimethylacrylamide, N-tert-butylacrylamide, acrylonitrile, methacrylonitrile. Cationic monomers of this group are, for example, dialkylaminoalkyl (meth)acrylates and dialkylaminoalkyl (meth)acrylamides such as dimethylaminomethyl acrylate, diethylaminoethyl acrylate, diethylaminoethyl methacrylate, and the salts of the monomers mentioned last with carboxylic acids or mineral acids, and also the quaternized products.

Further monomers are, for example, also monomers comprising hydroxyl groups, especially C₁-C₁₀-hydroxyalkyl (meth)acrylates, for example hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate.

Further monomers are phenyloxyethyl glycol mono(meth)acrylate, glycidyl acrylate, glycidyl methacrylate, amino(meth)acrylates such as 2-aminoethyl (meth)acrylate.

In particular, mixtures of the alkyl (meth)acrylates are also suitable.

Vinyl esters of carboxylic acids having from 1 to 20 carbon atoms are, for example, vinyl laurate, vinyl stearate, vinyl propionate, vinyl versatate and vinyl acetate.

Useful vinylaromatic compounds include vinyltoluene, α- and p-methylstyrene, α-butylstyrene, 4-n-butylstyrene, 4-n-decylstyrene, 2-vinylpryridine, N-vinylpyrrolidone and preferably styrene.

The vinyl halides are chlorine-, fluorine- or bromine-substituted ethylenically unsaturated compounds, preferably vinyl chloride, vinyl fluoride and vinylidene chloride.

Examples of vinyl ethers include methyl vinyl ether, ethyl vinyl ether, butyl vinyl ether, 4-hydroxybutyl vinyl ether, vinyl isobutyl ether or dodecyl vinyl ether. Preference is given to vinyl ethers of alcohols comprising from 1 to 4 carbon atoms.

Examples of aliphatic, optionally halogenated hydrocarbons having from 2 to 8 carbon atoms and 1 or 2 olefinic double bonds include ethylene, propene, isopropene, 1-butene, isobutene, butadiene, isoprene(2-methyl-1,3-butadiene) and chloroprene (2-chloro-1,3-butadiene).

It is also possible to use open-chain N-vinylamide compounds, for example N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide.

Examples of vinylidenes include vinylidene cyanide.

Further monomers are vinylacetic acid, vinylcarbazole, hydroxymethyl vinyl ketone, vinylene carbonate, tetrafluoroethylene, hexafluoropropene, nitroethylene, allylacetic acid, α-chloroacrylic esters, α-cyanoacrylic esters, methylenemalonic esters, α-cyanosorbic esters, cyclopentadiene and cyclopentene.

In addition to the main monomers mentioned and the inventive (meth)acrylic esters (F), the polymer may comprise further monomers, for example ethylenically unsaturated monomers with sulfonic acid or phosphonic acid groups, such as vinylsulfonic acid, allylsulfonic acid, styrenesulfonic acid, 2-acrylamidomethylpropanesulfonic acid or vinylphosphonic acid, allylphosphonic acid, styrenephosphonic acid, 2-acrylamido-2-methylpropanephosphonic acid.

In addition, all further monomers whose polymerization proceeds by a free-radically initiated mechanism are possible, as described, for example, in DE 100 41 211 A and in DE 101 48 497 A.

Further monomers also include crosslinking monomers.

It will be appreciated that it is also possible to use any mixtures of the main monomers mentioned for polymerization with at least one inventive (meth)acrylic ester (F). However, preference is given to polymerizing only one inventive (meth)acrylic ester (F) with at least one main monomer.

Preferred monomers are styrene, butadiene, acrylic acid, methacrylic acid, C₁-C₁₀-alkyl esters of acrylic acid and methacrylic acid, N-vinylpyrrolidone and acrylonitrile, and mixtures thereof.

It will be appreciated that it is also possible to polymerize the inventive (meth)acrylic esters (F) alone, so as to obtain homopolymers. In this case, preference is given to polymerizing only one (meth)acrylic ester (F).

A frequent method, but not the only method, for preparing such (co)polymers is free-radical or ionic (co)polymerization in a solvent or diluent.

The free-radical (co)polymerization of such monomers is effected, for example, in aqueous solution in the presence of polymerization initiators which decompose into free radicals under polymerization conditions, for example peroxodisulfates, H₂O₂ redox systems or hydroperoxides, for example tert-butyl hydroperoxide or cumene hydroperoxide. The (co)polymerization can be undertaken within a wide temperature range, if appropriate under reduced or else under elevated pressure, generally at temperatures up to 100° C. The pH of the reaction mixture is usually set within the range from 4 to 10.

The (co)polymerization may, though, also be performed in another manner known per se to those skilled in the art, continuously or batchwise, for example as a solution, precipitation, water-in-oil emulsion, inverse emulsion, suspension or inverse suspension polymerization.

In this case, the monomer(s) is/are (co)polymerized using free-radical polymerization initiators, for example azo compounds which decompose to free radicals, such as 2,2′-azobis(isobutyronitrile), 2,2′-azobis(2-amidinopropane) hydrochloride or 4,4′-azobis(4′-cyanopentanoic acid), or dialkyl peroxides such as di-tert-amyl peroxide, aryl alkyl peroxides such as tert-butyl cumyl peroxide, alkyl acyl peroxides such as tert-butyl peroxy-2-ethylhexanoate, peroxydicarbonates such as di(4-tert-butylcyclohexyl) peroxydicarbonate or hydroperoxides.

The compounds mentioned are usually used in the form of aqueous solutions or aqueous emulsions, the lower concentration being determined by the amount of water acceptable in the (co)polymerization and the upper concentration by the solubility of the compound in question in water.

The solvents or diluents used may, for example, be water, alcohols such as methanol, ethanol, n- or isopropanol, n- or isobutanol, or ketones such as acetone, ethyl methyl ketone, diethyl ketone or isobutyl methyl ketone. Particular preference is given to nonpolar solvents, for example xylene and their isomer mixtures, Shellsol® A and Solvent Naphtha.

In a preferred embodiment, the monomers are premixed, and initiator with any further additives are added dissolved in solvent. A particularly preferred embodiment is described in WO 2001/23484 and there particularly on page 10, line 3 to line 24.

If appropriate, the (co)polymerization can be performed in the presence of polymerization regulators, for example hydroxylammonium salts, chlorinated hydrocarbons and thio compounds, for example tert-butyl mercaptan, ethylacryloyl thioglycolate, mercaptoethynol, mercaptopropyltrimethoxysilane, dodecylmercaptan, tert-dodecyl mercaptan or alkali metal hypophosphites. In the (co)polymerization, these regulators may be used, for example, in amounts of from 0 to 0.8 part by weight, based on 100 parts by weight of the monomers to be (co)polymerized, by means of which the molar mass of the resulting (co)polymer is reduced.

In the emulsion polymerization, dispersants, ionic and/or nonionic emulsifiers and/or protective colloids or stabilizers may be used as interface-active compounds. Useful such compounds include both the protective colloids typically used for the performance of emulsion polymerizations and emulsifiers.

Suitable protective colloids are, for example, copolymers comprising polyvinyl alcohols, cellulose derivatives or vinylpyrrolidone. A comprehensive description of further suitable protective colloids can be found in Houben-Weyl, Methoden der organischen Chemie [Methods of organic chemistry], Volume XIV/1, makromolekulare Stoffe [macromolecular substances], Georg-Thieme-Verlag, Stuttgart, 1969, p. 411 to 420. It will be appreciated that it is also possible to use mixtures of emulsifiers and/or protective colloids. The dispersants used are preferably exclusively emulsifiers whose relative molecular weights, in contrast to the protective colloids, are typically below 1000. They may be of anionic, cationic or nonionic nature. It will be appreciated that, in the case of the use of mixtures of interface-active substances, the individual components must be compatible with one another, which can be checked in the case of doubt with reference to a few preliminary experiments. In general, anionic emulsifiers are compatible with one another and with nonionic emulsifiers.

The same also applies to cationic emulsifiers, while anionic and cationic emulsifiers are usually incompatible with one another. Common emulsifiers are, for example, ethoxylated mono-, di- and trialkylphenols (EO: 3 to 100, alkyl radical: O₄ to O₁₂), ethoxylated fatty alcohols (EO: 3 to 100, alkyl radical: C₈ to C₁₈), and alkali metal and ammonium salts of alkyl sulfates (alkyl radical: C₈ to C₁₈) of sulfuric monoesters of ethoxylated alkylphenols (EO: 3 to 100, alkyl radical: C₄ to O₁₂), of alkylsulfonic acids (alkyl radical: C₁₂ to C₁₈) and of alkylacryloylsulfonic acids (alkyl radical: C₉ to C₁₈). Further suitable emulsifiers such as sulfosuccinic esters can be found in Houben-Weyl, Methoden der organischen Chemie, Volume XIV/1, Makromolekulare Stoffe, Georg-Thieme Verlag, Stuttgart, 1961, pages 192 to 208.

In general, the amount of dispersant used is from 0.5 to 6% by weight, preferably from 1 to 3% by weight, based on the monomers to be polymerized by free-radical means.

The polymer dispersions in which (meth)acrylic esters (F) prepared in accordance with the invention are used may additionally be deodorized by chemical and/or physical means.

A chemical deodorization can be performed, for example, as disclosed by P.H.H. Araújo, C. Sayer, J. G. R. Poco, R. Giudici, in Polymer Engineering and Science, 2002 (42), 1442-1468, or in EP 1 375 530 B1.

The present application therefore further provides (co)polymers comprising the (meth)acrylic esters (F) obtainable by the process according to the invention.

The examples which follow are intended to illustrate the properties of the invention, but without restricting it.

### EXAMPLES

In this document, “parts” are understood to mean “parts by weight” unless stated otherwise.

### Example 1

### Preparation of Citronellyl Acrylate

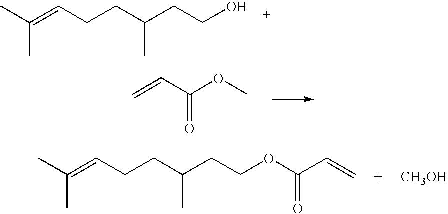

In a 4 l round-bottom flask with attached reflux condenser, 587 g of 3-citronellol (3.76 mol), 647 g of methyl acrylate (7.52 mol), 1128 g of 5 Å molecular sieve and 18.8 g of Novozym® 435 (supported lipase from Candida antarctica B, from Novozymes, Denmark) were mixed. The reaction mixture was stirred at 40° C. for 8 h. Thereafter, the enzyme and the molecular sieve were filtered off using a suction filter. The filtercake was washed with MTBE (tert-butyl methyl ether). The combined filtrate was concentrated on a rotary evaporator at 40° C. and 10 bar. 547 g (69% of the theoretical yield) of a slightly yellowish oil were obtained.

To determine the conversion, a sample was analyzed by means of GC. 99% of β-citronellol was converted to citronellyl acrylate, and no by-products whatsoever were formed.

### Example 2

### Reaction Profile of the Transesterification of Methyl Acrylate with β-citronellol

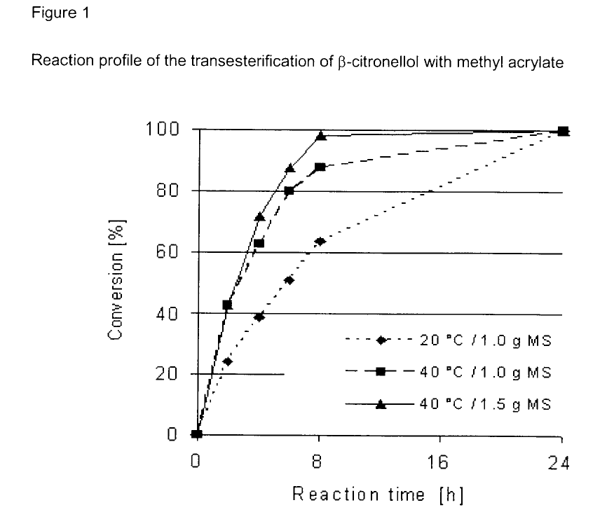

5 mmol of β-citronellol were mixed with 10 mmol of methyl acrylate, 25 mg of Novozym® 435 and 1.0 g or 1.5 g of 5 Å molecular sieve (MS), and shaken at 20 or 40° C. for 24 h. The conversion was determined by means of GC by sampling after 2, 4, 6, 8 and 24 h. The results are compiled in FIG. 1.

### Example 3

### Transesterification of Methyl Acrylate with Various Hydroxy-Functional Aromas

In each case 5 mmol of a hydroxy-functional aroma were mixed with 50 mmol of methyl acrylate, 50 mg of Novozym® 435 and 1.0 g of 5 Å molecular sieve (MS), and shaken in a waterbath at 40° C. for 24 h. The conversion was determined by means of GC by sampling after 6 and 24 h. The results are compiled in table 3.

TABLE 3
Transesterification of methyl acrylate with various hydroxy-functional aromas

| | | Conversion [%] of various hydroxy-functional aromas | | | | | |
|---|---|---|---|---|---|---|---|
| | | | | | Tetrahydro- | | |
| Solvent | Time [h] | Prenol | Geraniol | Hydroxyciol | geraniol | Morillol | Phytol |
| none | 6 | 100 | 100 | 100 | 100 | — | 99 |
| none | 24 | 100 | 100 | 100 | 100 | 62 | 98 |

### Example 4

### Copolymerization of Citronellyl Acrylate with N-Vinylpyrrolidone

The copolymerization of citronellyl acrylate with N-vinylpyrrolidone was performed in a 0.5 l stirred vessel with nitrogen feed and metering apparatus (feed 1 and 2). The nitrogen-purged initial charge comprised 83.30 g of ethanol (cosmetic), 5.00 g of citronellyl acrylate and in each case 10% of the amount of feed 1 and 2, and was preheated to 65° C. within 15 min. Feed 1 comprised 1.00 g of Wako® V-59 (2,2′-azobis(2-methylbutyronitrile)) and 75.00 g of ethanol (cosmetic). Feed 2 comprised 95.00 g of N-vinylpyrrolidone and 75.00 g of ethanol (cosmetic). Feed 2 was metered in within 4 h and feed 1 within 4.5 h. Subsequently, polymerization was continued at 68° C. for 1 h. After further addition of 2.0 g of Wako® V-59 in 50 g of ethanol within 30 min, polymerization was continued at 68° C. for another 8 h, then the mixture was cooled to room temperature and transferred. The solids content was 30.2% by weight based on the total weight of the dispersion.

### Example 5

### Copolymerization of Hydroxyciol Acrylate with N-Vinylpyrrolidone

The copolymerization of hydroxyciol acrylate with N-vinylpyrrolidone was performed in a 0.5 l stirred vessel with nitrogen feed and metering apparatus (feed 1 and 2). The nitrogen-purged initial charge comprised 83.30 g of ethanol (cosmetic), 10.00 g of hydroxyciol acrylate and in each case 10% of the amount of feed 1 and 2, and was preheated to 65° C. within 15 min. Feed 1 comprised 1.00 g of Wako® V-59 and 75.00 g of ethanol (cosmetic). Feed 2 comprised 95.00 g of N-vinylpyrrolidone and 75.00 g of ethanol (cosmetic). Feed 2 was metered in within 4 h and feed 1 within 4.5 h. Subsequently, polymerization was continued at 68° C. for 1 h. After further addition of 2.0 g of Wako® V-59 in 50 g of ethanol within 30 min, polymerization was continued at 68° C. for another 8 h, then the mixture was cooled to room temperature and transferred. The solids content was 31.2% by weight based on the total weight of the dispersion.

### Example 6

### Hydrolysis of Citronellyl Acrylate or Hydroxyciol Acrylate Copolymer and Determination of the β-citronellol or Hydroxyciol Released

In each case 0.5 g of acrylate copolymer according to example 4 or example 5 was dissolved in 2.5 g of water and 1.5 g of abs. ethanol. After the dissolution, the sample was in each case alkalized with 0.5 g of aqueous sodium hydroxide solution (10% by weight) and stirred at room temperature. After 21 days had passed, the sample was in each case neutralized with aqueous phosphoric acid (10% strength by weight), diluted to 10 ml with abs. ethanol and analyzed by means of infrared spectroscopy (decrease in the absorbance of the carboxylic ester band of the copolymerized acrylate at 1722 cm⁻¹, normalized to 10% solution on the basis of 1751/1703 cm⁻¹; analysis by means of Endurance ATR unit with single reflection and diamond window). The results are compiled in table 4.

TABLE 4
Determination of the β-citronellol or hydroxyciol released by
means of infrared spectroscopy

| | | IR | Proportion of |
|---|---|---|---|
| | IR | absorbance | fragrance alcohol |
| | absorbance | after | released |
| Example | before NaOH | NaOH | [%] |
| 4 | 0.00054 | 0 | 100 |
| 5 | 0.00118 | 0.0005 | 58 |

### Example 7

### Copolymerization of Citronellyl Acrylate with Itaconic Acid

The copolymerization of citronellyl acrylate with itaconic acid was performed in a 0.5 l stirred vessel with attached reflux condenser. The nitrogen-purged initial charge comprised 105.00 g of isopropanol, 52.50 g of citronellol acrylate and 48.75 g of itaconic acid, and was preheated to 85° C. Within 3.5 h, the feed consisting of 6.70 g of methyl ethyl ketone, 36.00 g of isopropanol and 0.45 g of Porofor® N (2,2′-azodiisobutyronitrile) was metered in. After the end of the feed, polymerization was continued for another 1 h, then the mixture was cooled to room temperature and transferred. The solids content was 40.8% by weight, based on the total weight of the dispersion.

### Example 8

### Hydrolysis of the Copolymer of Citronellyl Acrylate with Itaconic Acid

2.09 g of the polymer according to example 7 were weighed with 10.48 g of water and 6.28 g of ethanol. 40% sodium hydroxide solution was used to adjust the pH of the solution to 12-14. The mixture was stirred at 60° C. over several days. After 3 h and 21 h, a sample (approx. 2.5 g) was taken in each case and neutralized with aqueous o-phosphoric acid (85% strength by weight). Subsequently, the content of β-citronellol released was quantified by means of gas chromatography. The results are compiled in table 5.

TABLE 5

| Hydrolysis time | Content of β-citronellol | Proportion of fragrance |
|---|---|---|
| [h] | [g/100 g] | alcohol released [%] |
| 3 | 1.17 | 3 |
| 21 | 10.9 | 27 |

1. A process for preparing a (meth)acrylic ester (F) of hydroxy-functional aroma (A), comprising esterifying at least one hydroxy-functional aroma (A) in the presence of at least one enzyme (E) with (meth)acrylic acid (S), or transesterifying the at least one hydroxy-functional aroma (A) with at least one (meth)acrylic ester (D), wherein the transesterification is effected in the absence of solvents.

2. The process according to claim 1, wherein a hydroxyl group of the hydroxy-functional aroma (A) is primary.

3. The process according to claim, wherein the hydroxy-functional aroma (A) used is a primary alcohol of general formula (I): 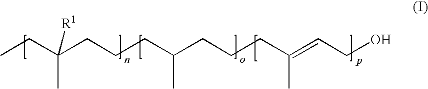
wherein n, o and p are each integers of from 0 to 10 in each case,
and at least one of the variables n, o or p is at least 1,
and wherein particular monomer units which are bracketed by the variables n, o and p are present in any sequence, and R¹ is selected from the group consisting of hydrogen, hydroxyl and C₁-C₁₀-alkyl.

4. The process according to claim 1, wherein the hydroxy-functional aroma is selected from the group consisting of citronellol, farnesol, geraniol, geranylgeraniol, hydroxyciol, phytol, prenol and tetrahydrogeraniol.

5. The process according to claim 1, wherein the hydroxyl group of the hydroxy-functional aroma (A) is secondary.

6. The process according to claim 5, wherein the hydroxy-functional aroma is selected from the group consisting of acetoin, menthol and morrilol.

7. The process according to claim 1, wherein the (meth)acrylic ester (D) is a saturated C₁-C₁₀-alkyl ester.

8. The process according to claim 1, wherein the (meth)acrylic ester (D) is selected from the group consisting of methyl (meth)acrylate, ethyl (meth)acrylate, n-butyl (meth)acrylate and 2-ethylhexyl (meth)acrylate.

9. The process according to claim 1, wherein the enzyme (E) is selected from the group consisting of the esterases (E.C. 3.1.-.-), lipases (E.C. 3.1.1.3), glycosylases (E.C. 3.2.-.-) and proteases (E.C. 3.4.-.-).

10. A (Meth)acrylic ester (F) of hydroxy-functional aroma (A), obtainable obtained by a process according to claim 1.

11. A monomer or comonomer in slow-release fragrance polymers, comprising the (Meth)acrylic ester (F) according to claim 10.
